(19) United States
(12) Patent Application Publication
(10) Pub. No.: US 2008/0095846 A1
(43) Pub. Date: Apr. 24, 2008

(54) Pharmaceutical compositions of antihistamine and decongestant

(76) Inventors: Gour Mukherji, Maharashtra (IN); Patil Jayadev, Mashrashtra (IN); sidharth Mate, Maharastra (IN)

Correspondence Address: BIO INTELLECTUAL PROPERTY SERVICES (BIO IPS) LLC, 8509 KERNON CT., LORTON, VA, 22079, US

(21) Appl. No.: 11/666,923

(22) PCT Filed: Nov. 4, 2004
(86) PCT No.: PCT/IB04/03612

§ 371 Date: May 3, 2007

(51) Int. Cl. A61K 9/24

(52) U.S. Cl. 424/472

(57) ABSTRACT

The present invention discloses a bilayer tablet composition comprising controlled or modified release decongestants such as pseudoephedrine or its salts or derivatives thereof with suitable pharmaceutical excipients with immediate release second layer comprising piperidinoalkanol compounds such as fexofenadine or its salts and derivatives thereof manufactured using just one functional excipient. The composition is useful particularly in the prophylaxis and treatment of allergic rhinitis.

### FIELD OF INVENTION

The present invention relates to pharmaceutical compositions comprising antihistamines, such as piperidinoalkanol compounds and decongestants such as pseudoephedrine and its salts and derivatives thereof. In particular, the present invention is concerned with pharmaceutical formulations comprising combinations of piperidinoalkanol compounds and pseudoephedrine, useful particularly in the prophylaxis and treatment of allergic rhinitis.

### BACKGROUND OF INVENTION

Rhinitis is a term describing the symptoms produced by nasal irritation or inflammation. It is a reaction that occurs in the eyes, nose and throat when airborne irritants (allergens) trigger the release of histamine. Symptoms of rhinitis include runny nose, itching, sneezing and stuffy nose due to blockage or congestion. These symptoms are the nose's natural response to inflammation and irritation.

Rhinitis lasting less than six weeks is called acute rhinitis, and persistent symptoms are called chronic rhinitis. Acute rhinitis is usually caused by infections or chemical irritation. Chronic rhinitis may be caused by allergy or a variety of other factors. There are two categories of allergic rhinitis—seasonal, i.e., it occurs particularly during pollen seasons. Symptoms of seasonal allergic rhinitis occur in spring, summer and/or early fall and are usually caused by allergic sensitivity to pollens from trees, grasses or weeds, or to airborne mold spores and perennial—i.e., occurs throughout the year. It's generally caused by sensitivity to house dust mites, animal dander and/or mold spores.

Antihistamines and decongestants are the most commonly used medications for allergic rhinitis. Antihistamines are the most inexpensive and commonly used treatment for rhinitis. It has been established that various piperidinoalkanol compounds are useful as antihistamines, antiallergy agents and bronchodilators as disclosed in U.S. Pat. No. 3,878,217. Antihistamines provide palliative relief, as rhinitis is a chronic, recurrent condition.

Antihistamines include sedative and non-sedative antihistamines. Examples of non-sedative antihistamines include terfenadine, loratadine, astemizole, fexofenadine etc. Of these, fexofenadiene, chemically (±)-4-[1-hydroxy-4-[4 (hydroxydiphenylmethyl)-1-piperidinyl]-butyl]-α,α-dimethyl benzeneacetic acid hydrochloride is commercially available in solid unit dosage forms for the treatment of seasonal allergic rhinitis. Decongestants are drugs that dry up nasal, sinus and bronchial secretions. They open sinus drains and help evacuate mucous from the sinus cavities. Pseudoephedrine is used as a nasal decongestant for temporary relief of nasal decongestion associated with upper respiratory allergy. The drug is also used to provide temporary relief of sinus, congestion and pressure. Pseudoephedrine and its salts when formulated as conventional solid dosage forms are prescribed orally three or four times daily for the relief of nasal congestion. Also, the modified release tablets of pseudoephedrine that releases the drug or its salts at a controlled release rate such that they may be administered once or twice daily are available commercially.

Antihistamines and decongestants are administered together for complete relief from rhinitis symptoms than therapy with either component alone. Antihistamines and decongestant products are often combined to relieve multiple symptoms of congestion and drainage and reduce the side effects of both products. Antihistamines produce sedation; decongestants are added to make them “non-drowsy.” The combined allergy product then relieves congestion and a running nose.

Pseudoephedrine or its salts are commonly administered with long acting antihistamines such that the antihistamine is released immediately in a conventional manner, and pseudoephedrine is released at a controlled rate in the body.

Many such compositions are described in literature.

U.S. Pat. No. 6,039,974 (Hoechst Marion Roussel) describes pharmaceutical composition in the form of a bilayer tablet comprising (a) a first discrete zone made up with a therapeutically effective decongestant amount of a sympathomimetic drug or its salt with carnauba wax and a suitable antiadherent, which provides sustained release of the sympathomimetic drug. The second discrete zone made up of piperidinoalkanol or a pharmaceutically acceptable salt in a second carrier base material comprising mixture of cellulose diluents in the preferable concentration of 27 to 73%, binder in the preferable concentration of 15 to 30%, disintegrant in the preferable concentration of 0.25 to 6% and lubricant in the preferable concentration of 0.25 to 2%. The said layer is prepared using aqueous wet granulation technique. The said layer provides an immediate release of the piperidinoalkanol or its pharmaceutically acceptable salt thereof. The said formulation presented comparative dissolution profile of piperidinoalkanol derivative with that of Allegra® whereas exhibited a slow release profile of pseudoephedrine hydrochloride compared to that of Sudafed®.

U.S. Pat. No. 6,267,986 (Ranbaxy Laboratories) relates to a process for the preparation of a controlled drug delivery system comprising two discrete zones wherein the first discrete zone comprises pseudoephedrine as active ingredient along with hydrophilic polymer and group II metal ion salts of polyuronic acid, such as, sodium calcium alginate, and the second discrete zone comprises a long-acting antihistamine selected from the group consisting of loratadine, azatidine, fexofenadine, terfenadine, cetirizine, astemizole and levocabastine or their pharmaceutically acceptable salt as active ingredient. The first layer comprising pseudoephedrine is manufactured by roll compaction process and the second layer comprising fexofenadine is manufactured by wet granulation technique.

U.S. Pat. No. 4,929,605 (Merrell Dow Pharmaceuticals) describes a pharmaceutical composition of piperidinoalkanol compounds only in solid unit dosage form comprising (a) a therapeutically effective amount of a piperidinoalkanol compound, (b) pharmaceutically acceptable nonionic or cationic surfactant in an amount of from about 0.1% to about 6% by weight of the composition and (c) pharmaceutically acceptable carbonate salt in an amount of from about 2% to about 50% by weight of the composition. The said composition is manufactured using wet granulation technique.

U.S. Pat. No. 4,996,061 (Merrell Dow Pharmaceuticals) discloses a pharmaceutical composition in the form of tablet comprising of two discrete zones. The first discrete zone is made with formulation (A) comprising a therapeutically effective decongestant amount of a sympathomimetic drug, one or more pharmaceutically acceptable water-soluble non-ionic cellulose ethers in an amount from about 18% to about 50% by weight of formulation (A), and one or more pharmaceutically acceptable excipients. The second discrete zone is made with formulation (B) comprising a therapeutically effective antihistaminic amount of a piperidinoalkanol, calcium carbonate in an amount from about 0.5% to about 25% by weight of formulation (B), one or more pharmaceutically acceptable anionic surfactants in an amount from about 1% to about 10% by weight of formulation (B), and one or more pharmaceutically acceptable excipients. Formulation (B) may optionally contain a therapeutically effective decongestant amount of a sympathomimetic drug. The said formulation showed cracking and unacceptable physical strength of tablets on final compression.

U.S. Pat. No. 4,999,226 (Merrell Dow Pharmaceuticals) relates to a multi-layered tablet containing an ibuprofen layer, a piperidinoalkanol antihistamine layer, and a layer or layers containing conventional pharmaceutical excipients, which is interspersed between the ibuprofen and piperidinoalkanol layer and serves to physically separate them. This tablet solves the problems associated with the physical and chemical incompatibilities between ibuprofen and the piperidinoalkanol antihistamines.

PCT application WO 03/084510 (Dr Reddy's Laboratories), published on Oct. 16, 2003 relates to the pharmaceutical composition for antihistaminic-decongestant combination in the form of unit dosage form. One of the preferred embodiments of the invention is directed towards the use of novel polymorph of fexofenadine with a least one decongestant in the form of bilayered tablet and process of making such bilayered tablets. The preferred polymorphs of fexofenadiene hydrochloride are polymorph A and polymorph X. The fexofenadine layer is prepared using wet granulation by using nonaqueous solvents and the pseudoephedrine layer is prepared using direct compression technique. The use of nonaqueous solvents may be advantageous as it can reduce drying time. Yet, the primary disadvantage of nonaqueous solvents is their flammability, which means expensive safety precautions and flameproof equipments are required. Further, fexofenadine layer comprises carrier base material comprising a mixture of filler in the preferable concentration of 30 to 45%, disintegrant in the preferable concentration of 6 to 10% and lubricant and/or glidant in the preferable concentration of 0.2 to 3%. The said mixture is then compressed with decongestant granules to a bilayer tablet. The said formulation has an in-vitro profile similar to that of the reference product i.e. AllegraD®.

PCT application PCT/IB/2004/002099 (Wockhardt Co.) relates to the pharmaceutical composition for piperidinoalkanol derivatives such as fexofenadine or salts, solvates and derivatives thereof. The application describes fexofenadine composition comprising fexofenadine with single functional excipient. The formulation is prepared by aqueous wet granulation technique.

It will be appreciated from prior art discussed above that many compositions for the combination product have been described in the prior art.

Despite the availability of different compositions, there is a clinical need for better preparations that are simple, stable and can confer an unswerving dissolution profile and are manufactured by an expedient manufacturing process. To this end, it is surprisingly found that a pharmaceutical composition for antihistaminic-decongestant combination according to the present invention provides an enhanced, synergistic therapeutic effect in terms of treatment of allergic rhinitis and related disorders. The said combination formulation is made up of two discrete zones the first layer comprising at least one decongestant releasing slowly over a specific period of time and the second layer comprising of immediate release antihistamine along with just one functional excipient.

Particularly, the granulation of antihistamine part of the composition is prepared either by dry compaction method or wet granulation with aqueous and/or hydroalcoholic solvents, The wet granulation using only one functional excipient thus brings the advantage of simple composition with easy process. Also, the piperidinoalkanol derivatives being minimally soluble in water, the choice of process and the therapeutically inactive ingredients becomes very important as the said composition is expected to allow efficient and immediate absorption for antihistamine and good bioavailability for both the components after oral administration thereof.

### SUMMARY OF THE INVENTION

The present invention provides an oral solid pharmaceutical combination composition comprising of two distinctive layers of which one layer comprises of a therapeutically effective amount of a piperidinoalkanol compound or a pharmaceutically acceptable salt thereof, and the individual isomers thereof with at least one functional excipient preferably diluents used in wet granulation process along with suitable binder. Disintegrants, glidants and lubricants are added extra granular to the drug granules. The other layer comprises of therapeutically effective amount of decongestant such as pseudoephedrine and its salts and derivatives thereof along with suitable excipients in an extended release form that releases the drug over specified period of time and a suitable process of manufacturing the same. Particularly, the antihistamine part of the composition is prepared either by dry granulation or wet granulation with aqueous and/or hydroalcoholic solvents.

Accordingly, it is an object of the present invention to provide a pharmaceutical composition and a process of manufacturing the same for the combination of long acting antihistamine drugs with decongestant such as pseudoephedrine, its salts and derivatives thereof.

The present invention, therefore also provides a pharmaceutical formulation comprising at least one piperidinoalkanol compound or a pharmaceutically acceptable salt thereof in therapeutically effective amount along with at least one functional excipient preferably diluents used in granulation comprising suitable binder, followed by blending of extragranular disintegrants and glidants and lubricants in either granulated or powder state, in one layer and the other layer comprising of therapeutically effective amount of at least one decongestant such as pseudoephedrine and its salts and derivatives thereof along with suitable excipients in an extended release form that releases the drug over controlled period of time. Alternatively, the piperidinoalkanol containing composition is processed by dry granulation of a blend containing the piperidinoalkanol drug and other suitable excipients, followed by compression to form one layer and the other layer comprises of the decongestant composition.

The present invention further provides a method of administering to a subject in need of treatment a pharmaceutical product or formulation substantially as hereinbefore described and in particular a bilayer composition which can typically be administered twice daily, and as such is particularly suited for the treatment of allergic rhinitis.

The present invention further provides use of at least one long acting antihistamine, and at least one decongestant, in the manufacture of a medicament for the treatment of allergic rhinitis. Such a medicament according to the present invention comprises a formulation substantially as herein described, and in particular a bilayer formulation, typically a bilayer tablet formulation substantially as hereinafter described.

Another aspect of the invention is process of manufacturing of the said composition according to the present invention. The formulation according to the present invention comprises of two distinctive layers of which first layer comprises of at least one decongestant such as pseudoephedrine and its salts and derivatives thereof is prepared by wet granulation and the second layer comprising of long acting antihistamine such as fexofenadine and its salts and derivatives thereof, manufactured either by (i) blending together a mixture of therapeutically active agent and at least one functional excipient, preferably, diluent or mixture of diluents before granulation with a binder solution; thus avoiding the use of disintegrant of any kind in the granulation blend followed by dry blending of other tablet excipients or (ii) dry granulation of drug with suitable tablet pharmaceutical excipients for an immediate drug release, followed by compression process and finally the tablet thus prepared is optionally coated further to yield consistent quality product. The resultant formulation has an in-vitro dissolution and biopharmaceutical profile similar to the reference product, Allegra-D®.

The present formulation process provides obvious benefits being simple operational process for manufacturing said oral bilayered pharmaceutical composition.

### BRIEF DESCRIPTION OF THE DRAWINGS

FIG. 1 illustrates comparative dissolution profile of Fexofenadine hydrochloride from Fexofenadine hydrochloride 60 mg and Pseudoephedrine hydrochloride 120 mg tablet (Example—1) versus Allegra-D® Tablets.

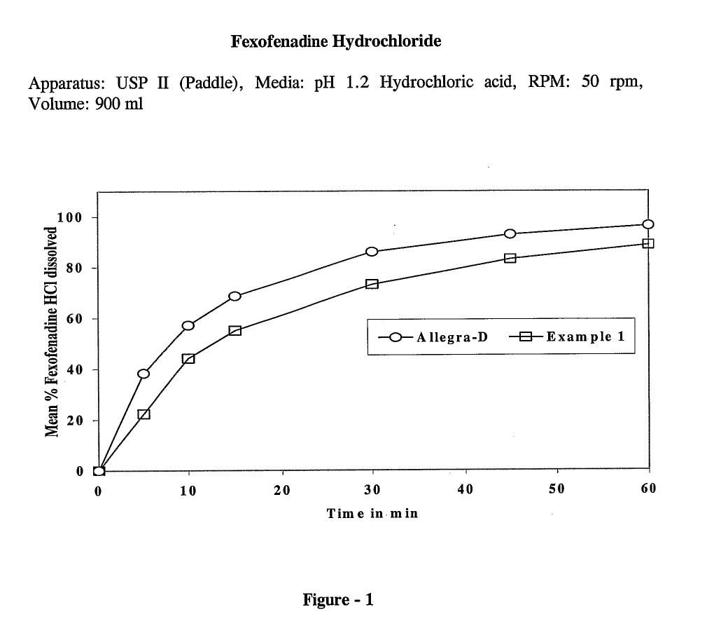

FIG. 2 illustrates comparative dissolution profiles of Pseudoephedrine hydrochloride from Fexofenadine hydrochloride 60 mg and Pseudoephedrine hydrochloride 120 mg tablet (Example—1) versus Allegra-D® Tablets.

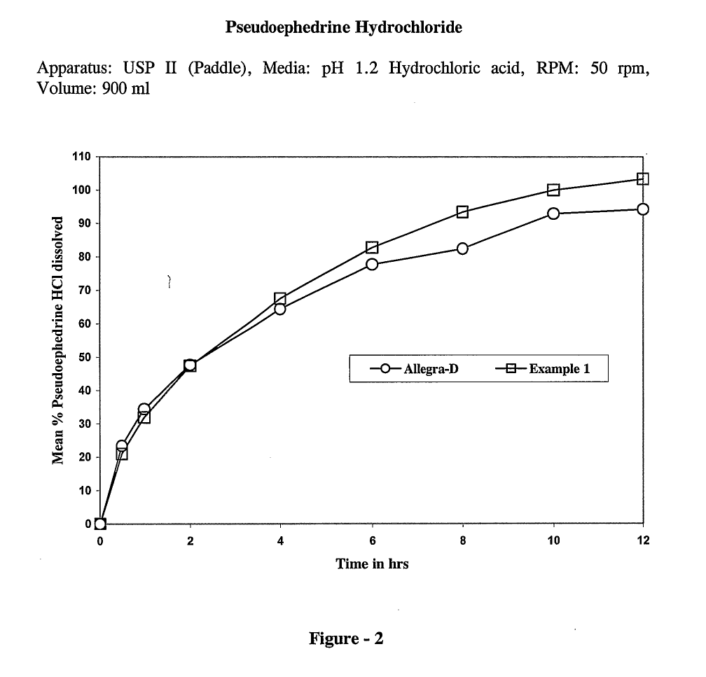

FIG. 3 illustrates comparative dissolution profiles of Fexofenadine hydrochloride from Fexofenadine hydrochloride 60 mg and Pseudoephedrine hydrochloride 120 mg tablet (Example—2) versus Allegra-D® Tablets.

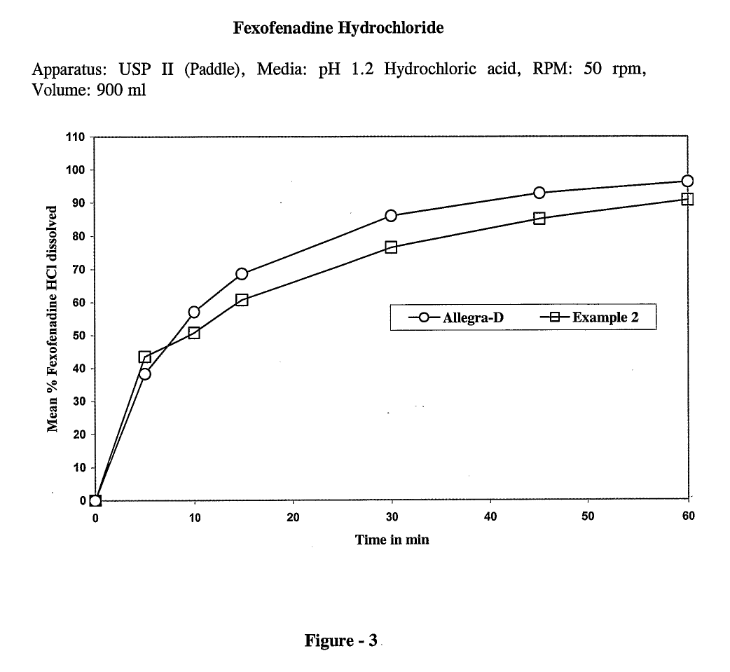

FIG. 4 illustrates comparative dissolution profiles of Pseudoephedrine hydrochloride from Fexofenadine Hydrochloride 60 mg and Pseudoephedrine hydrochloride 120 mg tablet (Example—2) versus Allegra-D® Tablets

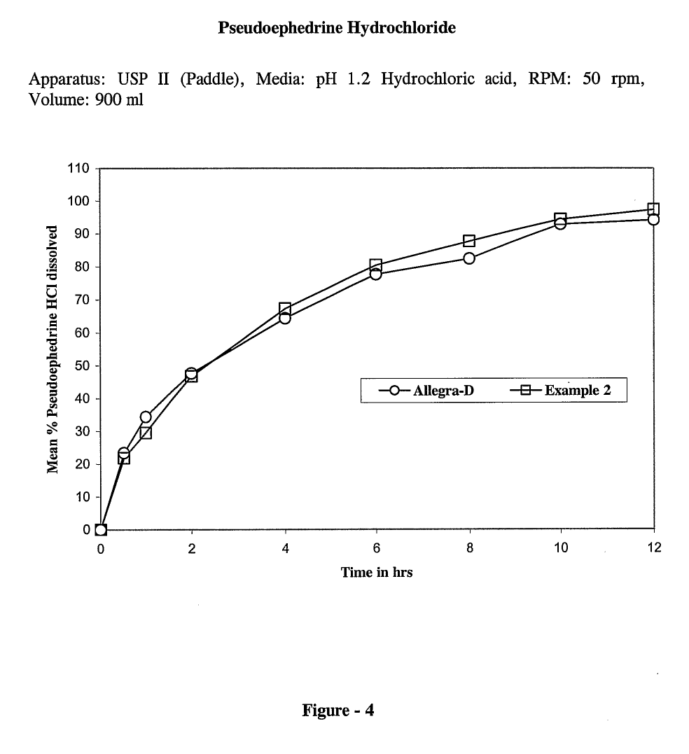

FIG. 5 illustrates comparative dissolution profiles of Fexofenadine hydrochloride from Fexofenadine Hydrochloride 60 mg and Pseudoephedrine hydrochloride 120 mg tablet (Example—3) versus Allegra-D® Tablets

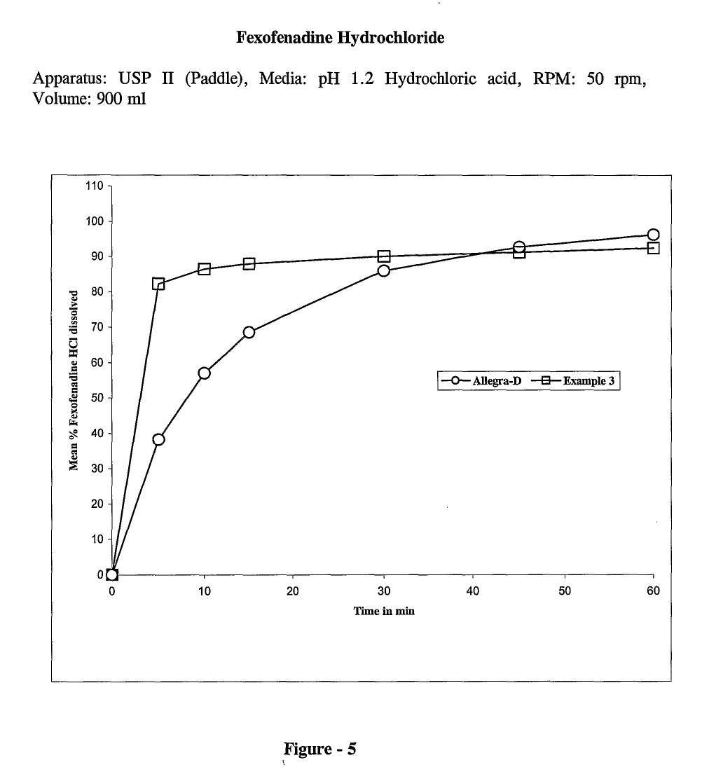

FIG. 6 illustrates comparative dissolution profiles of Pseudoephedrine hydrochloride from Fexofenadine Hydrochloride 60 mg and Pseudoephedrine hydrochloride 120 mg tablet (Example—3) versus Allegra-D® Tablets

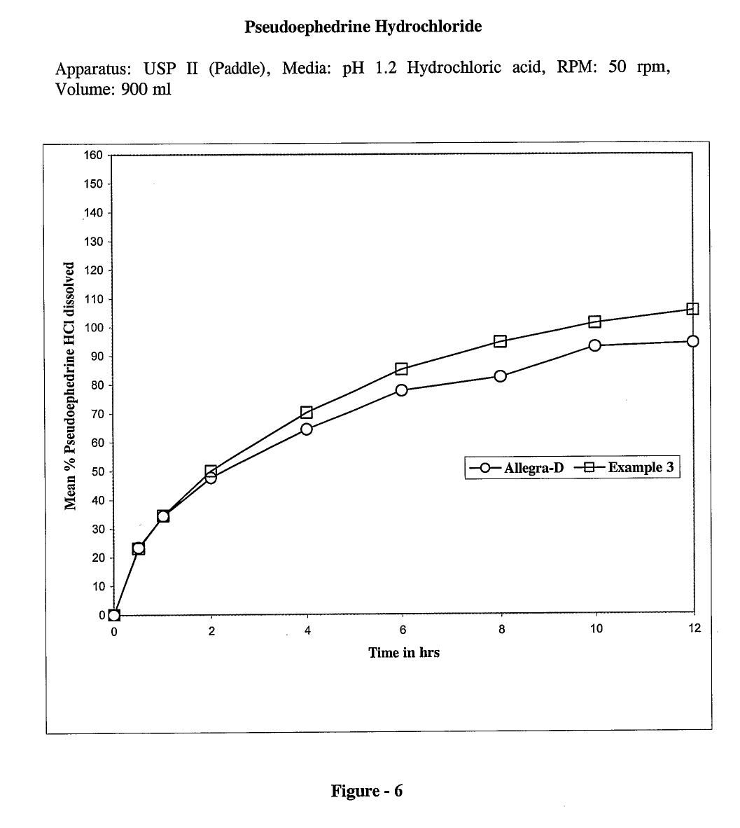

### DETAILED DESCRIPTION OF THE INVENTION

The present invention provides an oral solid pharmaceutical combination composition comprising of two distinctive layers of which one layer comprises of a therapeutically effective amount of a piperidinoalkanol compound or a pharmaceutically acceptable salt thereof, and the individual isomers thereof along with suitable excipients in an immediate release form and the other layer comprising of therapeutically effective amount of decongestant such as pseudoephedrine and its salts and derivatives thereof along with suitable excipients in an extended release form that releases the drug over controlled period of time.

The term “functional excipient” is defined as a single excipient or mixture of excipients, which have similar functions. For example, the use of lactose or calcium carbonate or a mixture of lactose and calcium carbonate and a mixture of lactose and microcrystalline cellulose falls within the scope of the definition whereas a mixture of lactose/calcium carbonate with croscarmellose sodium/starch or a mixture of lactose/croscarmellose sodium does not fit within the scope of the definition as they possess different functionalities.

The term “dry granulation” describes dry method of granulation where the primary powder particles are aggregated under high pressure. There are two main processes. Either a “slug” is produced under heavy-duty tabletting press (called as slugging) or the powder is squeezed between two rollers to produce a sheet of material (called as roll compaction). In both cases these intermediate products are broken using suitable milling technique to produce granular material, which is sifted and mixed with lubricants prior to compression.

In particular, a formulation according to the present invention provides a sustained or controlled release source of at least one decongestant and an immediate release source of at least one long acting antihistamine. Preferably the controlled release source of at least one decongestant such as pseudoephedrine and its salts and derivatives thereof comprises a formulation core comprising at least one decongestant and further comprises or is provided with a water soluble or water swellable polymeric release material typically provided as a matrix to the formulation core. The bilayer tablet is optionally coated for obvious benefits of the process to obtain quality product.

In a preferred embodiment of the present invention, a time-specific controlled release or sustained release formulation comprises bilayer system including the pharmaceutically active agents effective for the treatment of allergic rhinitis and related disorders, with a water-soluble or water-swellable polymer matrix in the first layer along with controlled release pseudoephedrine and its salts and derivatives thereof. More particularly, a bilayer formulation according to the present invention comprises a first layer comprising a formulation core comprising decongestant such as pseudoephedrine and its salts and derivatives thereof and further comprising or being provided with a water-soluble or water swellable polymeric release material. The said polymeric material delays the release of the drug from the first layer over a period of 12 hours. The second immediate release layer comprising long acting antihistamine such as fexofenadine and its salts and derivatives thereof along with suitable diluents, binder, disintegrants and lubricants.

Suitably the decongestant containing formulation core of a pharmaceutical formulation according to the present invention may be formulated so as to allow the release of the drug there from in a still further controlled or sustained release manner, subsequent to the desired lag-time provided by the inclusion of a water soluble or water swellable polymeric material. It will be appreciated, therefore, that conventional core excipients may be employed in decongestant containing layer according to the present invention; alternatively, excipients which are capable of forming a sustained release matrix system may be used.

In particular, the present invention provides tablet formulation (suitably compressed bilyer tablet formulations) comprising an immediate release source of at least one long acting antihistamine and a sustained or controlled release source of at least one decongestant.

Preferably the sustained release source of at least one decongestant comprises a formulation layer comprising at least one decongestant provided with a water-soluble or water swellable polymer. A preferred decongestant for use in a tablet according to the present invention is pseudoephedrine and its salts and derivatives thereof and a preferred long acting antihistamine is fexofenadine and its salts and derivatives thereof. As such in a formulation according to the present invention, pseudoephedrine hydrochloride, after oral administration, can be released in a sustained manner independent of pH and fexofenadine hydrochloride can be released immediately.

A bilayer tablet according to the present invention comprises a combination of materials, including for example one layer comprising one or more water-soluble or water swellable hydrophilic polymers, diluents and lubricants and other layer of immediate release fexofenadine comprising at least one functional excipient preferably diluents used in wet granulation followed by blending comprising additional diluents, disintegrants and optionally lubricants, in either powdered or in separately granulated forms or mixtures thereof

The above materials are combined in specific concentration with therapeutically effective amounts of active agents to achieve the immediate release and extended release characteristics of the respective layers in the said tablet formulation according to the present invention.

In general, the present invention provides a process for the manufacture of a pharmaceutical product. The immediate release layer is prepared by using wet granulation or dry granulation by roll compaction processes whereas the controlled release layer is prepared using wet granulation technique.

One process for preparing antihistamine layer comprises blending of fexofenadine hydrochloride with at least one suitable functional excipient such as diluent before wet granulation with a binder solution; prepared by dissolving binder in suitable aqueous and/or alcoholic or hydroalcoholic solvents, thus avoiding the use of intragranular disintegrant of any kind. Some of the remaining tablet excipients in the composition is separately blended and then optionally compacted using suitable roller compacter. The compacts are milled, sized and lubricated and blended with the drug granules. More specifically it is believed that by roller compacting a blend prepared in accordance with the current invention, the granule property of the blend improves. The blend of the active pharmaceutical ingredient and the excipients in the granular state influences the granule size ranges, bulk density and the dissolution properties of the active pharmaceutical ingredient. This approach provides the means to develop a reproducible process for manufacture of fexofenadine layer in the present invention.

Alternatively, second process for preparing antihistamine layer comprises blending of fexofenadine hydrochloride with tablet excipients such as diluents, disintegrants, glidants and lubricants and the like and then compacting using suitable roll compactor. The compacts are milled, sized and lubricated to obtain desired granules and then compressed with pesudoephedrine granules on a bilayer machine. The bilayer tablet thus manufactured can be optionally coated to get quality product. More specifically, the present invention comprises the use of a roller compaction process to form consistent granules, which provides a consistent dissolution profile among various lots of dosage formulation blends comprising fexofenadine hydrochloride. It is believed that by roller compacting a blend prepared in accordance with the current invention, the ingredients are forced into a state of intimate contact, mixing and adhesion. The particles undergo rearrangement, and it is believed that particle fracturing creates multiple surface sites, contact points and bonding sites between the active pharmaceutical ingredient and the excipients; this enhanced contact between the drug and the excipients improves physical properties of the granules thus obtained.

Thus the antihistamine layer prepared by using fexofenadine and just one functional excipient, preferably a diluent or mixture of diluents and devoid of any disintegrant at the granulation stage, results in a formulation that gives the desired in-vitro dissolution profile of fexofenadine or its salts, solvates and derivatives thereof.

Suitable excipients employed in a pharmaceutical formulation according to the present invention, may include commonly used pharmaceutical excipients such as microcrystalline cellulose, silicified microcrystalline cellulose (Prosolv SMCC 90®), dibasic calcium phosphate, lactose and various forms of lactose, hydroxypropyl methylcellulose, powdered cellulose, carboxymethyl cellulose, hydroxyethyl cellulose, and other cellulose derivatives, magnesium oxide, calcium carbonate, magnesium aluminum silicate and other mineral bases.

The artisan can further select appropriate water-soluble or water swellable polymers available in the literature. Most preferably, polymers used are hydroxypropyl methylcellulose, carboxymethyl cellulose, hydroxyethyl cellulose, and other cellulose derivatives, povidone, gum and gum derivatives, gelatin, polymethacrylates and its derivatives thereof.

The formulation according to present invention may also include pharmaceutically acceptable lubricants and glidants. The artisan can select appropriate lubricants and glidants in order to get good flow to aid in compression of the tablets.

Examples of suitable lubricants and glidants include magnesium stearate, calcium stearate, glyceryl behenate, light mineral oil, polyethylene glycol, sodium stearyl fumarate, stearic acid, talc, hydrogenated vegetable oil, calcium silicate, magnesium silicate and colloidal silicon dioxide. The most preferred pharmaceutical lubricant and glidants are talc, silicon dioxide and magnesium stearate.

The present invention further comprises pharmaceutical composition having two distinctive layers of which first layer comprises of a therapeutically effective amount of decongestant or a pharmaceutically acceptable salt, solvate or derivatives thereof along with at least one carrier material comprising a mixture of at least one controlled release polymer in concentration from about 40 to 80 percent by weight of first layer preferably from about 50 to 75 percent by weight of first layer; and a suitable filler or diluent in concentration from about 4 to 20 percent weight of first layer, preferably from about 4 to 15 percent by weight of first layer wherein said carrier material provides a controlled or modified release of the decongestant drug; and the second layer comprises of therapeutically effective amount of piperidinoalkanol compound or a pharmaceutically acceptable salt, solvate or derivative or individual isomers thereof with at least one functional excipient such as filler or diluent or mixture of diluents in the concentration from 20 to 80 percent by weight of second layer preferably from about 25 to 70 percent by weight of second layer optionally with a suitable surfactant in the concentration from 0.1 to 3 percent by weight of second layer preferably from about 0.3 to 1.5 percent by weight of second layer wherein the said second layer provides immediate release of piperidinoalkanol compound or a pharmaceutically acceptable salt, solvate or derivative or individual isomers thereof.

The present invention further comprises a process of preparing a pharmaceutical product, or a pharmaceutical formulation, or a medicament substantially as hereinbefore described. Suitably such process comprises providing at least one long acting antihistamine such as fexofenadine hydrochloride and salts, solvates and derivatives thereof and at least one decongestant such as pseudoephedrine hydrochloride and its salts, solvates and derivatives thereof, and formulating at least one long acting antihistamine and at least one decongestant. Suitably such a process comprises providing an immediate release source of the at least one long acting antihistamine, and a sustained or controlled release source of at least one decongestant, wherein preferably the formulation comprises a bilayer pharmaceutical formulation, typically in tablet form.

Substantially as hereinbefore described the tablets of the present invention comprise bilayer tablets, of which, a first layer comprising a decongestant, such as pseudoephedrine hydrochloride, is prepared by mixing pseudoephedrine hydrochloride, diluents and polymers. The resulting mixture may then be blended and wet granulated with a binder such as hydroxypropyl cellulose or xanthan gum. The granulate may then be sized through sieves of optimum mesh, mixed with glidants and lubricants. The resulting mixture may then be compressed.

Preferably a second layer comprising antihistamine such as fexofenadine hydrochloride may be prepared by mixing fexofenadine hydrochloride with diluent, before wet granulation with a binder solution; prepared by dissolving binder in suitable aqueous and/or hydroalcoholic solvents, thus avoiding the use of intragranular disintegrant of any kind. Some of the remaining ingredients like disintegrants, glidants and lubricants and diluents may be optionally blended and compacted using suitable roller compactor. The compacts may then be sized through a sieve of optimum mesh, and mixed with the drug granules together with lubricants. Alternatively, the fexofenadine is blended with extragranular excipients such as diluents, disintegrants, glidants and lubricants and the like and compacted to form granules and then compressed with pesudoephedrine granules on a bilayer machine. The tablet thus manufactured may be optionally coated.

The present invention will now be further illustrated by the following examples, which does not limit the scope of the invention in any way. Further different strengths of the formulation may be achieved by proportionally using a dose weight formula. The artisan can also vary the concentrations of excipients to achieve the desired dissolution profile.

### EXAMPLES

### Example—1

| Sr. No. | Ingredients | Quantity (mg/Tablet) |
|---|---|---|
| A) Immediate release layer | | |
| 1. | Fexofenadine hydrochloride anhydrous | 60.0 |
| 2. | Lactose monohydrate | 117.5 |
| 3. | Povidone K-30 | 9.0 |
| 4. | Polysorbate-80 | 2.0 |
| 5. | Prosolv SMCC 90* | 70.0 |
| 6. | Croscarmelose sodium (Ac-di-sol) | 85.0 |
| 7. | Talc | 5.0 |
| 8. | Colloidal silicon dioxide | 2.0 |
| 9. | Stearic acid | 4.50 |
| 10. | Purified Water | q.s. |
| B) Controlled release layer | | |
| 11. | Pseudoephedrine hydrochloride | 120.0 |
| 12. | Microcrystalline cellulose | 32.9 |
| 13. | Hydroxyethyl cellulose (Natrosol | 65.0 |
| | 250M) | |
| 14. | Hydroxypropyl methylcellulose | 250.0 |
| | (Methocel K15M) | |
| 15. | Iron oxide red | 0.6 |
| 16. | Xanthan gum | 3.0 |
| 17. | Purified water | q.s. |
| 18. | Magnesium stearate | 5.5 |
| 19. | Colloidal silicon dioxide | 3.0 |
| C) Film Coating | | |
| 20. | Opadry YS - IR-7006 Clear | 24.0 |
| 21. | Purified water | q.s. |

Process: A) Immediate Release Layer:

Blend fexofenadine hydrochloride with lactose monohydrate. Prepare binder solution by dissolving Povidone K-30 and Polysorbate-80 in purified water. Granulate the blend of drug and diluent with this binder solution. Dry the granules and sift using sieve of suitable mesh size. Compact a blend comprising Prosolv SMCC 90 and Ac-di-sol using a roll compactor, then mill and sift the compacts using sieve of suitable mesh size. Blend the two granule fractions and lubricate the resultant granules with talc, stearic acid and colloidal silicon dioxide.

B) Controlled Release Layer:

Blend pseudoephedrine hydrochloride with hydroxyethyl cellulose and hydroxypropyl methylcellulose. Add microcrystalline cellulose and iron oxide red to the drug blend and mix. Sift the mixture using sieve of suitable mesh size. Prepare binder solution by dissolving xanthan gum in purified water. Granulate the blend with this binder solution. Dry the granules and sift using sieve of suitable mesh size. Lubricate the dried granules with colloidal silicon dioxide and magnesium stearate.

Compress the granules of both the layers using bilayer compression machine into bilayer tablets.

Opadry (HPMC) clear aqueous solution is used to coat the bilayer tablets.

### Example—2

| Sr. No. | Ingredients | Quantity (mg/Tablet) |
|---|---|---|
| A) Immediate release layer | | |
| 1. | Fexofenadine hydrochloride anhydrous | 60.0 |
| 2. | Lactose monohydrate | 119.5 |
| 3. | Povidone K-30 | 9.0 |
| 4. | Prosolv SMCC 90* | 90.0 |
| 5. | Croscarmelose sodium (Ac-di-sol) | 75.0 |
| 6. | Talc | 5.0 |
| 7. | Colloidal silicon dioxide | 2.0 |
| 8. | Stearic acid | 4.5 |
| 9. | Purified Water | q.s. |
| B) Controlled release layer | | |
| 10. | Pseudoephedrine hydrochloride | 120.0 |
| 11. | Microcrystalline cellulose | 63.9 |
| 12. | Hydroxyethyl cellulose (Natrosol | 115.0 |
| | 250M) | |
| 13. | Hydroxypropyl methylcellulose | 170.0 |
| | (Methocel K15M) | |
| 14. | Hydroxypropyl cellulose (Klucel L F) | 7.0 |
| 15. | Iron oxide red | 0.6 |
| 16. | Purified water | q.s. |
| 17. | Magnesium stearate | 5.5 |
| 18. | Colloidal silicon dioxide | 3.0 |
| C) Film Coating | | |
| 19. | Opadry YS - IR-7006 Clear | 30.0 |
| 20. | Purified water | q.s. |
| 21. | Isopropyl alcohol | q.s. |

Process: A) Immediate Release Layer:

Blend fexofenadine hydrochloride with lactose monohydrate. Prepare binder solution by dissolving Povidone K-30 in purified water. Granulate the blend of drug and diluent with this binder solution. Dry the granules and sift using sieve of suitable mesh size. Compact a blend comprising Prosolv SMCC 90 and Ac-di-sol using a roll compactor, mill and sift the compacts using sieve of suitable mesh size, Blend the two fractions of granules and lubricate the resultant granules with talc, stearic acid and colloidal silicon dioxide.

B) Controlled Release Layer:

Blend pseudoephedrine hydrochloride with hydroxyethyl cellulose and hydroxypropyl methylcellulose. Add microcrystalline cellulose and iron oxide red to the drug blend and mix. Sift the mixture using sieve of suitable mesh size. Prepare binder solution by dissolving Klucel LF in purified water. Granulate the blend with this binder solution. Dry the granules and sift using sieve of suitable mesh size. Lubricate the dried granules with colloidal silicon dioxide and magnesium stearate.

Compress the granules of both the layers using bilayer compression machine into bilayer tablets.

Opadry (HPMC) clear hydroalcoholic solution is used to coat the bilayer tablets.

### Example—3

| Sr. No. | Ingredients | Quantity (mg/Tablet) |
|---|---|---|
| A) Immediate release layer | | |
| 1. | Fexofenadine hydrochloride anhydrous | 60.0 |
| 2. | Microcrystalline cellulose | 127.0 |
| 3. | Prosolv SMCC 90* | 70.0 |
| 4. | Croscarmelose sodium (Ac-di-sol) | 75.0 |
| 5. | Talc | 8.0 |
| 6. | Colloidal silicon dioxide | 3.0 |
| 7. | Stearic acid | 7.0 |
| 8. | Purified Water | q.s. |
| B) Controlled release layer | | |
| 9. | Pseudoephedrine hydrochloride | 120.0 |
| 10. | Microcrystalline cellulose | 32.9 |
| 11. | Hydroxyethyl cellulose (Natrosol | 65.0 |
| | 250M) | |
| 12. | Hydroxypropyl methylcellulose | 250.0 |
| | (Methocel K15M) | |
| 13. | Xanthan Gum | 3.0 |
| 14. | Iron oxide red | 0.6 |
| 15. | Purified water | q.s. |
| 16. | Magnesium stearate | 5.5 |
| 17. | Colloidal silicon dioxide | 3.0 |
| C) Film Coating | | |
| 18. | Opadry YS - IR-7006 Clear | 30.0 |
| 19. | Purified water | q.s. |

Process: A) Immediate Release Layer:

Blend fexofenadine hydrochloride with microcrystalline cellulose, Prosolv SMCC-90, Ac-di-sol, talc, colloidal silicon dioxide and stearic acid. Compact this blend using a roll compactor, mill and then sift the compacts using sieve of suitable mesh size. Repeat the process to get uniform granules of desired yield.

B) Controlled Release Layer:

Blend pseudoephedrine hydrochloride with hydroxyethyl cellulose and hydroxypropyl methylcellulose. Add microcrystalline cellulose and iron oxide red to the drug blend and mix. Sift the mixture using sieve of suitable mesh size. Prepare binder solution by dissolving xanthan gum in purified water. Granulate the blend with this binder solution. Dry the granules and sift using sieve of suitable mesh size. Lubricate the dried granules with colloidal silicon dioxide and magnesium stearate.

Compress the granules of both the layers using bilayer compression machine into bilayer tablets.

Opadry (HPMC) clear aqueous solution is used to coat the bilayer tablets.

1-48. (canceled)

49. A pharmaceutical composition comprising two distinctive layers wherein the first layer comprises a therapeutically effective amount of a decongestant or a pharmaceutically acceptable salt, solvate or a derivative thereof, along with a carrier material comprising a mixture of at least one controlled release polymer and a suitable filler or a diluent, wherein the carrier material provides a controlled or modified release of the decongestant drug; and the second layer comprises a therapeutically effective amount of a long acting antihistamine or a pharmaceutically acceptable salt, solvate or a physiologically acceptable derivative or individual isomer thereof with at least one functional excipient selected from one or more of a filler, or a diluent, optionally with a suitable surfactant in a concentration from about 0.1 to about 3 percent by weight of the second layer, wherein the second layer provides an immediate release of the long acting antihistamine or a pharmaceutically acceptable salt, solvate or a physiologically acceptable derivative or individual isomer thereof.

50. The pharmaceutical composition of claim 49, wherein the long acting antihistamine is selected from the group consisting of terfenadine, loratadine, astemizole and fexofenadine or their pharmaceutically acceptable salts, solvates, physiologically acceptable derivatives or isomers thereof.

51. The pharmaceutical composition of claim 50, wherein the long acting antihistamine is fexofenadine hydrochloride or a pharmaceutically acceptable salt, solvate, physiologically acceptable derivative or isomer thereof.

52. The pharmaceutical composition of claim 49, wherein the decongestant is pseudoephedrine hydrochloride.

53. The pharmaceutical composition of claim 49, wherein the diluent comprises one or more of microcrystalline cellulose, silicified microcrystalline cellulose, lactose and derivatives, powdered cellulose & other cellulose derivatives, starch, and starch derivatives.

54. The pharmaceutical composition of claim 49, wherein the immediate release layer further comprises one or more of binders, disintegrants, surfactants, glidants and lubricants.

55. The pharmaceutical composition of claim 49, wherein the immediate release layer is prepared by a process comprising aqueous wet granulation or compaction.

56. The pharmaceutical composition of claim 55, wherein the wet granulation process comprises aqueous or hydroalcoholic granulation.

57. The pharmaceutical composition of claim 49, wherein the pharmaceutical composition is in the form of a bilayer tablet.

58. A process for the preparation of a pharmaceutical composition in the form of a tablet comprising two distinctive layers, wherein the first layer provides a controlled release of a decongestant and the second layer provides an immediate release of a long acting antihistamine and wherein the immediate release layer is prepared by a process comprising: (a) mixing a long acting antihistamine drug and one or more diluents; (b) blending using a suitable blender; (c) granulating the blend to form granules; (d) mixing the granules and blending with one or more of diluents, disintegrants, lubricants, and glidants; and (e) compressing with the controlled release layer using a bilayer tabletting machine.

59. The process of claim 58, wherein the controlled release layer comprising a decongestant is prepared by a process comprising direct compression or compaction.

60. The process of claim 59, wherein the process comprises mixing a decongestant with one or more of a water soluble or water swellable polymer, disintegrant, glidant, and a lubricant.

61. The process of claim 59, wherein the decongestant is pseudoephedrine hydrochloride.

62. The process of claim 60, wherein the water soluble or water swellable polymer comprises one or more of polyvinylpyrrolidone, hydroxypropylmethylcellulose, hydroxyethylcellulose, methyl cellulose, sodium carboxymethylcellulose, hydroxypropylcellulose, and polyacrylates.

63. The process of claim 58, wherein the tablet may optionally be coated using suitable coating polymers.

64. The process of claim 58, wherein the tablet is in the form of a bilayer tablet.
